(19) United States
(12) Patent Application Publication
(10) Pub. No.: US 2026/0000630 A1
(43) Pub. Date: Jan. 1, 2026

(54) PHARMACEUTICAL COMPOSITION COMPRISING SALBUTAMOL

(71) Applicant: APTAR FRANCE SAS, LE NEUBOURG (FR)

(72) Inventors: Isabelle PEYRON, Castres (FR); Irene ROSSI, San Benedetto del Tronto (Ascoli Piceno) (IT)

(21) Appl. No.: 18/992,711

(22) PCT Filed: Jul. 11, 2023
(86) PCT No.: PCT/FR2023/051079

§ 371 Date: Jan. 9, 2025

(30) Foreign Application Priority Data
Jul. 13, 2022 (FR) FR2207237

(51) Int. Cl. A61K 31/137; A61K 9/00; A61K 47/10

(52) U.S. Cl. CPC A61K 31/137; A61K 9/008; A61K 47/10

(57) ABSTRACT

The present invention relates to a pharmaceutical composition consisting of an active ingredient based on salbutamol (a), 1,1-difluoroethane (b), a polyethylene glycol (c) and ethanol (d).

The invention also relates to the use of such a pharmaceutical composition in the treatment of respiratory disorders, to a canister comprising it as well as to a metered-dose inhaler provided with such a canister.

Finally, the invention relates to the uses of this pharmaceutical composition and of this canister in a metered-dose inhaler.

### TECHNICAL FIELD

The present invention relates to a pharmaceutical composition which is capable of being used in the treatment of respiratory disorders and which comprises an active ingredient based on salbutamol, in particular salbutamol sulphate, and a propellant gas formed by 1,1-difluoroethane.

The invention also relates to a canister comprising this pharmaceutical composition as well as to a metered-dose inhaler provided with such a canister.

The invention finally relates to the uses of this pharmaceutical composition and of this canister in a metered-dose inhaler.

### PRIOR ART

Salbutamol as well as its derivatives are active ingredients known as bronchodilators in the treatment of respiratory disorders such as asthma and chronic obstructive pulmonary diseases (COPD).

Pharmaceutical compositions comprising salbutamol or one of its derivatives are conventionally delivered to patients by means of a metered-dose inhaler (MDI).

A metered-dose inhaler is an administration device equipped with a canister containing the pharmaceutical composition, a metering valve allowing to distribute a controlled quantity of pharmaceutical composition containing the active ingredient and an applicator allowing to exert a pressure on the metering valve and provided with a mouthpiece.

The pharmaceutical composition comprises a propellant gas in which the active ingredient is dissolved, suspended or dispersed, and optionally one or more other compounds that can be in particular chosen from surfactants, polar excipients and preservatives.

The choice of the propellant gas implemented in the metered-dose inhalers for pharmaceutical purposes has changed over the years.

Given their detrimental effects on the ozone layer, chlorofluorocarbons (CFC), used for a long time, were abandoned in favour of hydrofluoroalkanes (HFA) such as 1,1,1,2-tetrafluoroethane (HFA-134a or R-134a) and 1,1,1,2,3,3,3-heptafluoropropane (HFA-227ea or R-227ea), which are compounds that do not have a harmful effect on the ozone layer or human toxicity.

However, since these hydrofluoroalkanes R-134a and R-227ea are characterised by a high global warming potential (GWP) with a non-negligible impact on the greenhouse effect, pharmaceutical compositions comprising salbutamol, or one of its derivatives, and an alternative propellant gas have been proposed.

Thus, documents WO 2013/054137 A1, WO 2014/170689 A1 and WO 2013/054135 A1, respectively referred to as [1] to [3] in the rest of the present description, describe the implementation of a specific hydrofluorocarbon (HFC) as an alternative propellant gas, 1,1-difluoroethane (HFC-152a or R-152a) in pharmaceutical compositions comprising salbutamol sulphate.

More particularly, the pharmaceutical compositions described in documents [1] and [2] comprise salbutamol sulphate, R-152a as well as one or more surfactants, the role of which is to aid in the dispersion of the particles of active ingredient in the propellant gas.

In document [1], at least one of the surfactants is oleic acid.

Advantageously, the pharmaceutical compositions of document [1] only comprise salbutamol sulphate, R-152a and oleic acid, in the absence of ethanol. This document [1] specifies that the choice of R-152a as propellant gas and of oleic acid as surfactant allows to obtain pharmaceutical compositions that, even in the absence of ethanol, have good therapeutic performance when they are delivered using a drug administration device such as a metered-dose inhaler.

On the contrary, in document [2], at least one of the surfactants is not oleic acid. This surfactant can in particular be chosen from ethyl oleate, polyvinylpyrrolidone (PVP), sorbitan monooleate, sorbitan trioleate, isopropyl myristate, the polyethylene glycols such as polyethylene glycol 300, polyoxyethylene 20 sorbitan monooleate or monolaurate, propoxylated polyethylene glycol and lecithin, with it being specified that the preferred surfactant is PVP.

Advantageously, the pharmaceutical compositions described in document [2] comprise salbutamol sulphate, R-152a and one or more surfactants but do not contain any oleic acid. Preferably, these pharmaceutical compositions also do not contain any ethanol while preserving good therapeutic performance when they are delivered using a device of the metered-dose inhaler (MDI) type.

Contrary to the pharmaceutical compositions described in documents [1] and [2], the pharmaceutical compositions described in document [3] do not comprise any surfactant for the reason that surfactants would not be desirable and that it would be of interest to form a stable suspension without resorting to their implementation. Document [3] specifies that the use of the propellant gas R-152a allows to prepare pharmaceutical compositions which are not only free of surfactants but also free of polar excipients and which have good therapeutic performance when they are delivered using a device of the metered-dose inhaler (MDI) type.

However, experience shows that the aerosolisation performance of pharmaceutical compositions described in these documents [1] to [3], and more particularly in documents [1] and [3], is not satisfactory.

It is observed in particular that pharmaceutical compositions consisting of salbutamol sulphate, R-152a and oleic acid according to the teaching of document [1] are characterised by aerosolisation performance that is much lower than that of pharmaceutical compositions according to the teaching of document [3], which are however free of surfactants, even though the role of these surfactants is to aid in the dispersion of the particles of active ingredient (salbutamol sulphate) and to stabilise it in the propellant gas (R-152a).

Moreover, the aerosolisation performance of these pharmaceutical compositions described in documents [1] to [3] decreases over time, which is detrimental to their therapeutic effectiveness.

It is therefore on the basis of these observations and in order to continually improve the aerosolisation properties and, consequently, the therapeutic properties conferred by the pharmaceutical compositions intended for the treatment of respiratory disorders that the present invention is based.

### DISCLOSURE OF THE INVENTION

These goals and others are reached, first of all, by a pharmaceutical composition of the aforementioned type, that is to say which comprises an active ingredient based on salbutamol (a) and 1,1-difluoroethane (b).

According to the invention, the pharmaceutical composition consists of the following compounds:
(a) an active ingredient based on salbutamol,
(b) 1,1-difluoroethane (R-152a),
(c) a polyethylene glycol, and
(d) ethanol.

The inventors have observed that, in an unexpected and surprising manner, a pharmaceutical composition that consists of an active ingredient based on salbutamol (a), 1,1-difluoroethane (b), a polyethylene glycol (c) and ethanol (d) allows to achieve much better aerosolisation performance than the pharmaceutical compositions of documents [1] to [3], which all contain salbutamol and 1,1-difluoroethane but are preferably free of ethanol, whether these pharmaceutical compositions comprise a surfactant such as oleic acid like in document [1] or are free thereof like in document [3].

As illustrated in the examples below, the pharmaceutical composition according to the invention is furthermore characterised by good aerosolisation performance that is stable over time and, consequently, by a therapeutic effect that performs well and is long-lasting.

The pharmaceutical composition according to the invention comprises an active ingredient (a) based on salbutamol.

In an advantageous variant of the pharmaceutical composition according to the invention, this active ingredient (a) is a pharmaceutically acceptable salt of salbutamol.

In a preferred variant of the invention, this active ingredient (a) is salbutamol sulphate.

The active ingredient (a) is advantageously in the form of particles, the size of which is adapted to delivery by inhalation of the pharmaceutical composition in which it is contained. Conventionally, the median diameter of the particles of active ingredient (a) is less than or equal to 5 μm and, preferably, between 0.5 μm and 4 μm.

In a variant of the composition according to the invention, the mass proportion of active ingredient (a) is between 0.05% and 0.5% by mass relative to the total mass of the pharmaceutical composition. This mass proportion is advantageously between 0.1% and 0.4% by mass and, preferably, between 0.2% and 0.35% by mass relative to the total mass of the pharmaceutical composition.

The pharmaceutical composition according to the invention also comprises 1,1-difluoroethane (b) as propellant gas.

In a variant of the composition according to the invention, the mass proportion of 1,1-difluoroethane (b) is between 87.5% and 99.89% by mass relative to the total mass of the pharmaceutical composition.

In another variant, the mass proportion of 1,1-difluoroethane (b) is between 92.5% and 99.89% by mass relative to the total mass of the pharmaceutical composition. This mass proportion is advantageously between 95.6% and 99.68% by mass and, preferably, between 97.15% and 99.26% by mass relative to the total mass of the pharmaceutical composition.

The pharmaceutical composition according to the invention also comprises polyethylene glycol (c) as surfactant.

Polyethylene glycol (c) or PEG is a linear polyether synthesised from monomers of ethylene oxide and corresponding to the formula H—(CH₂—CH₂—O)ₙ—OH, n being an integer such that n≥4.

In a variant of the composition according to the invention, the polyethylene glycol (c) has a mass average molar mass M_{w} less than or equal to 20000 g/mol. This mass average molar mass M_{w} of the PEG is advantageously between 100 g/mol and 5000 g/mol and, preferably, between 200 g/mol and 2000 g/mol.

In particular, PEGs having a mass average molar mass M_{w} of 400 g/mol, 600 g/mol and 1000 g/mol, respectively designated by the acronyms PEG 400, PEG 600 and PEG 1000, are particularly suitable for the pharmaceutical composition according to the invention.

In a variant of the composition according to the invention, the mass proportion of polyethylene glycol (c) is between 0.01% and 2% by mass relative to the total mass of the pharmaceutical composition. This total mass proportion is advantageously between 0.02% and 1% by mass and, preferably, between 0.04% and 0.5% by mass relative to the total mass of the pharmaceutical composition.

Finally, the pharmaceutical composition according to the invention comprises ethanol (d) as polar excipient.

In a variant of the composition according to the invention, the mass proportion of ethanol (d) is between 0.05% and 10% by mass relative to the total mass of the pharmaceutical composition.

In another variant, the mass proportion of ethanol (d) is between 0.05% and 5% by mass relative to the total mass of the pharmaceutical composition.

The aerosolisation performance of the pharmaceutical composition according to the invention can be achieved with a relatively low mass proportion of ethanol, which does not present any danger for the health of the patient, even young.

The mass proportion of ethanol (d) can advantageously be between 0.2% and 3% by mass and, preferably, between 0.5% and 2% by mass relative to the total mass of the pharmaceutical composition.

The present invention relates, secondly, to a pharmaceutical composition for a use in the treatment of patients suffering or liable to suffer from respiratory disorders.

According to the invention, this pharmaceutical composition, which is used in the treatment of respiratory disorders, is as defined above, that is to say that it consists of the following compounds:
(a) an active ingredient based on salbutamol,
(b) 1,1-difluoroethane (R-152a),
(c) a polyethylene glycol, and
(d) ethanol.

The features described above in relation to the pharmaceutical composition and, in particular, the features relative to the various compounds (a), (b), (c) and (d) forming this pharmaceutical composition and to their respective mass proportions are of course applicable to the present use in the treatment of respiratory disorders.

Such respiratory disorders can be asthma or chronic obstructive pulmonary diseases (COPD).

In the context of the present invention, the patients can be treated by the administration of a quantity, effective from a therapeutic point of view, of a pharmaceutical composition as defined above.

The present invention relates, thirdly, to a canister comprising a pharmaceutical composition as well as to a metered-dose inhaler provided with such a canister.

According to the invention, this pharmaceutical composition is as defined above, that is to say that it consists of the following compounds:
(a) an active ingredient based on salbutamol,
(b) 1,1-difluoroethane (R-152a),
(c) a polyethylene glycol, and
(d) ethanol.

Like above, the features relative to each of these compounds (a) to (d) can be taken alone or in combination.

The present invention relates, fourthly, to the use of a pharmaceutical composition and/or of a canister as defined above in a metered-dose inhaler (MDI), such a device being conventionally used to deliver pharmaceutical compositions comprising an active ingredient based on salbutamol or on a pharmaceutically acceptable salt thereof.

Other features and advantages of the invention will be clearer upon reading the following additional description, which relates to examples of pharmaceutical compositions as well as to the evaluation of their in vitro aerosolisation performance, two of the pharmaceutical compositions being pharmaceutical compositions according to the invention, noted as C and C′, the three others being comparative pharmaceutical compositions according to the teachings of documents [1] and [3], noted as C_{[1]}, C_{[1]}′ and C_{[3]}.

### BRIEF DESCRIPTION OF THE DRAWINGS

FIG. 1 shows the graphs illustrating the deposited fraction of particles of salbutamol (expressed in %) coming from five doses of pharmaceutical compositions C, C′, C_{[1]}, C_{[1]}′ and C_{[3]} as obtained at T0, as a function of the stages of the NGI pharmaceutical impactor, the pharmaceutical compositions C and C′ being according to the invention and the three others being comparative pharmaceutical compositions, the compositions C_{[1]} and C_{[1]}′ being according to the teaching of document [1] and the composition C_{[3]} being according to the teaching of document [3].

FIG. 2 shows the graphs illustrating the deposited fraction of particles of salbutamol (expressed in %) coming from five doses of the pharmaceutical compositions C, C′, C_{[1]}, C_{[1]}′ and C_{[3]} as obtained at T3M, as a function of the stages of the NGI pharmaceutical impactor.

FIG. 3 shows the graphs illustrating the deposited fraction of particles of salbutamol (expressed in %) coming from five doses of the pharmaceutical composition C according to the invention at T0 and at T3M, as a function of the stages of the NGI pharmaceutical impactor.

FIG. 4 shows the graphs illustrating the deposited fraction of particles of salbutamol (expressed in %) coming from five doses of the pharmaceutical composition C′ according to the invention at T0 and at T3M, as a function of the stages of the NGI pharmaceutical impactor.

### DETAILED DISCLOSURE OF SPECIFIC EMBODIMENTS

Five pharmaceutical compositions C, C′, C_{[1]}, C_{[1]}′ and C_{[3]} were prepared from the following compounds:
salbutamol sulphate as active ingredient (a),
R-152a as propellant gas (b),
polyethylene glycol (c) having a molecular mass of 400 g/mol (PEG 400) or oleic acid as surfactant, and/or
ethanol (d) as polar excipient.

The metered-dose inhalers that were the subject of the tests were prepared with the same batches of compounds, of canisters made of aluminium and of metering valves, according to an identical operating protocol.

In a first step, a metering valve was crimped onto each of the canisters with a suitable piece of equipment.

In a second step, five distinct series of metered-dose inhalers were filled, in two steps, by introducing via the metering valve:
a concentrated suspension comprising 30.125 mg of salbutamol sulphate, a reduced quantity of R-152a propellant gas and, if necessary, the mass proportion(s) of surfactant (PEG 400 or oleic acid) and of ethanol as indicated in table 1 below, relative to the total mass of pharmaceutical composition, then
a quantity of R-152a propellant gas sufficient to reach a total mass of pharmaceutical composition of 9.57 g.

TABLE 1

| Composition | C | C′ | C_{[1]} | C_{[1]}′ | C_{[3]} |
|---|---|---|---|---|---|
| PEG 400 (% by mass) | 0.05 | 0.1 | 0 | 0 | 0 |
| Oleic acid (% by mass) | 0 | 0 | 0.03 | 0.05 | 0 |
| Ethanol (% by mass) | 1.00 | 1.00 | 0 | 0 | 0 |

Two series of tests of measurement of aerodynamic particle size distribution (APSD) were carried out.

These two series of tests, which involve evaluating the aerodynamic size of the particles of active ingredient exiting the valve, were carried out via a multi-stage pharmaceutical impactor allowing an approximate modelling of the bronchial tree. In the present case, the pharmaceutical impactor used was the Next Generation Impactor (NGI), which corresponds to apparatus E of the European Pharmacopoeia.

More particularly, these two series of tests were carried out at a flow rate of 30 L/min, by expulsing 5 doses of each of the pharmaceutical compositions into the NGI impactor.

A first series of tests of measurement of aerodynamic particle size distribution were carried out on the pharmaceutical compositions C, C′, C_{[1]}, C_{[1]}′ and C_{[3]} as obtained at TO, that is to say after the step of filling in two steps described above. The results of this first series of tests are reported in FIG. 1.

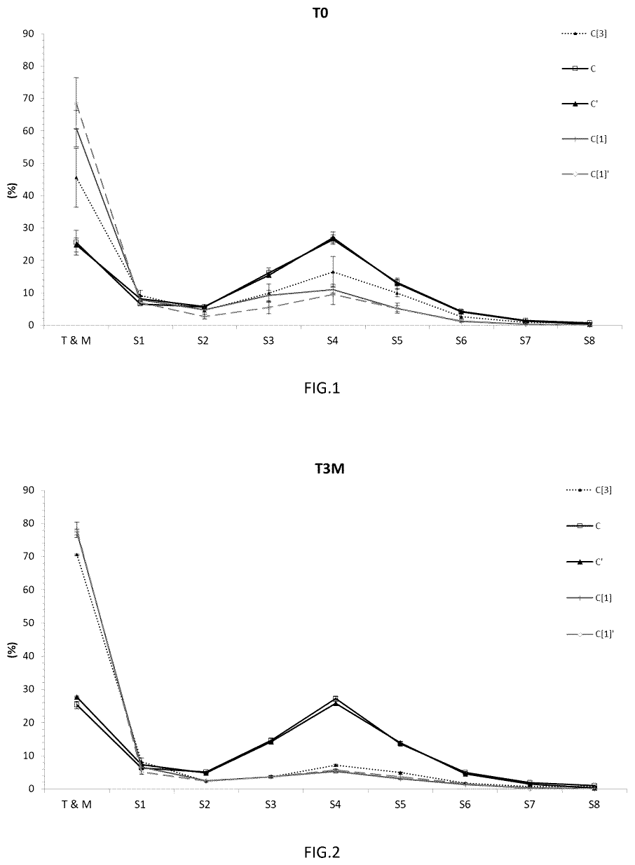

A second series of tests of measurement of aerodynamic particle size distribution were carried out on these same pharmaceutical compositions C, C′, C_{[1]}, C_{[1]}′ and C_{[3]} as obtained at T3M, that is to say after a storage having a duration of three months of the metered-dose inhalers comprising said compositions C, C′, C_{[1]}, C_{[1]}′ and C_{[3]}, these metered-dose inhalers being disposed in an inverted position (valve downwards) for these three months. The results of this second series of tests are reported in FIG. 2.

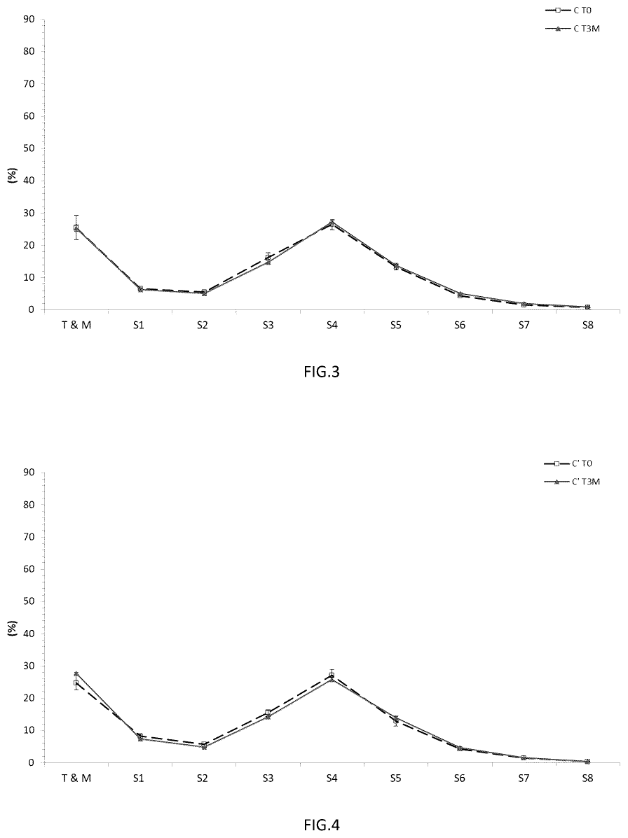

In addition, the results of the first and second series of tests carried out on the pharmaceutical compositions C and C′ according to the invention are reported in FIG. 3 for the composition C and in FIG. 4 for the composition C′.

The graphs of FIGS. 1 to 4 show the fractions of salbutamol (a) deposited, on the one hand, in the throat and in the mouth (noted as T&M) and, on the other hand, on each of the eight stages of the impactor (noted as S1 to S8).

To ensure an optimal therapeutic effectiveness, the fraction deposited in the throat and the mouth T&M must be minimised while the fractions deposited on the stages S3 to S5 must on the contrary be maximised.

FIG. 1 shows that the pharmaceutical compositions C and C′ according to the invention effectively allow to optimise this therapeutic effect.

On the one hand, it is observed that the fraction of salbutamol deposited using the compositions C and C′ at the level T&M is approximately 25%. This fraction is much smaller than the fractions of salbutamol that are deposited at this same level T&M using the comparative pharmaceutical compositions C_{[1]}, C_{[1]}′ and C_{[3]}, fractions that are respectively approximately 68%, 60% and 46%.

On the other hand, it is observed that the sum of the fractions of salbutamol deposited on the stages S3 to S5 using the pharmaceutical compositions C and C′ according to the invention is much greater than the sum of the fractions of salbutamol deposited on these same stages S3 to S5 using the comparative pharmaceutical compositions C_{[1]}, C_{[1]}′ and C_{[3]}. This observation is even more marked with the comparative compositions C_{[1]} and C_{[1]}′ which, however, use a surfactant, in this case oleic acid, the role of which is to aid in the dispersion of the particles of active ingredient and to stabilise them in the propellant gas.

Moreover, it is observed that the pharmaceutical compositions C and C′, which respectively include a mass proportion of 0.05% and 0.1% of PEG 400, are characterised by a performance in terms of therapeutic effectiveness that is indeed comparable, insofar as their respective graphs in FIG. 1 are superimposed at the level T&M and over the eight stages S1 to S8 of the impactor. Thus, even a reduced quantity of PEG in the pharmaceutical composition according to the invention allows to achieve an excellent therapeutic effectiveness.

FIG. 2 shows that the pharmaceutical compositions C and C′ according to the invention conserve this optimised therapeutic effectiveness after three months of storage. On the contrary, it is observed that this therapeutic effectiveness is highly degraded for all of the comparative pharmaceutical compositions C_{[1]}, C_{[1]}′ and C_{[3]}.

FIGS. 3 and 4 confirm that the pharmaceutical compositions C and C′ according to the invention indeed effectively conserve this same excellent therapeutic effectiveness after three months of storage. Indeed, the graphs of these FIGS. 3 and 4 are practically superimposable, reflecting the fact that the fraction of salbutamol deposited using the compositions C and C′ at the level T&M is similar or identical at T0 and at T3M, just like the sum of the fractions of salbutamol deposited on the stages S3 to S5 using these same pharmaceutical compositions C and C′ at T0 and T3M.

### BIBLIOGRAPHY

[1] WO 2013/054137 A1
[2] WO 2014/170689 A1
[3] WO 2013/054135 A1

What is claimed is:

1. Pharmaceutical composition consisting of the following compounds:
(a) an active ingredient based on salbutamol,
(b) 1,1-difluoroethane (R-152a),
(c) a polyethylene glycol, and
(d) ethanol.

2. Pharmaceutical composition according to claim 1, wherein the active ingredient (a) is a pharmaceutically acceptable salt of salbutamol.

3. Pharmaceutical composition according to claim 2, wherein the active ingredient (a) is salbutamol sulphate.

4. Pharmaceutical composition according to claim 1, wherein the polyethylene glycol (c) has a mass average molar mass M_{w} less than or equal to 20000 g/mol, advantageously between 100 g/mol and 5000 g/mol and, preferably, between 200 g/mol and 2000 g/mol.

5. Pharmaceutical composition according to claim 1, wherein the mass proportion of active ingredient (a) is between 0.05% and 0.5% by mass, advantageously between 0.1% and 0.4% by mass and, preferably, between 0.2% and 0.35% by mass relative to the total mass of the pharmaceutical composition.

6. Pharmaceutical composition according to claim 1, wherein the mass proportion of 1,1-difluoroethane (b) is between 87.5% and 99.89% by mass, in particular between 92.5% and 99.89% by mass, advantageously between 95.6% and 99.68% by mass and, preferably, between 97.15% and 99.26% by mass relative to the total mass of the pharmaceutical composition.

7. Pharmaceutical composition according to claim 1, wherein the mass proportion of polyethylene glycol (c) is between 0.01% and 2% by mass, advantageously between 0.02% and 1% by mass and, preferably, between 0.04% and 0.5% by mass relative to the total mass of the pharmaceutical composition.

8. Pharmaceutical composition according to claim 1, wherein the mass proportion of ethanol (c) is between 0.05% and 10% by mass, in particular between 0.05% and 5% by mass, advantageously between 0.2% and 3% by mass and, preferably, between 0.5% and 2% by mass relative to the total mass of the pharmaceutical composition.

9. Pharmaceutical composition according to claim 1, for a use in the treatment of respiratory disorders, such as asthma and chronic obstructive pulmonary diseases (COPD).

10. Canister comprising a pharmaceutical composition according to claim 1.

11. Metered-dose inhaler provided with a canister according to claim 10.

12. Use of the pharmaceutical composition according to claim 1, or of the canister according to claim 10 in a metered-dose inhaler.
